(19) United States
(12) Patent Application Publication
(10) Pub. No.: US 2004/0040556 A1
(43) Pub. Date: Mar. 4, 2004

(54) Baby pacefier with gentle supplemental oxygen feed

(76) Inventor: Thomas Eric Fillyaw, Cooper City, FL (US)

Correspondence Address: John H. Oltman, Oltman, Flynn & Kubler, 915 Middle River Drive #415, Ft. Lauderdale, FL, 33304-3585, US

(21) Appl. No.: 10/233,200

(22) Filed: Sep. 3, 2002

(51) Int. Cl. A61M 15/00

(52) U.S. Cl. 128/202.16

(57) ABSTRACT

A baby pacifier that provide supplemental oxygen to premature babies and other infants requiring such includes a housing that housing includes a front surface adapted to be symmetrically disposed about the baby's head at the mouth and nose areas, and a resilient nipple. The housing has an interior chamber into which supplemental oxygen is directed through an oxygen inlet opening of a barbed elbow or stem into the interior chamber. The oxygen inlet opening is adapted to be coupled to the external oxygen source using an external oxygen supply conduit. A plurality of oxygen delivery holes extend from the interior chamber through the housing about the baby's mouth and nose areas to allow supplemental oxygen to flow outwardly from the interior chamber. The nipple pacifies the baby during suckling while centering the baby pacifier about the baby's head at the mouth and nose areas. The baby pacifier may include a medication administering sponge to which a medication solution may be added such that when supplemental oxygen passes therethrough, the medication solution is gradually evaporated delivering medication vapors to the baby's nose area.

### BACKGROUND OF THE INVENTION

1. Field of the Invention

The present invention relates generally to the field of devices that provide supplemental oxygen to premature babies and other infants requiring such. More specifically the present invention relates to a baby pacifier for insertion into the mouth of a baby for gently feeding supplemental oxygen supplied from an external oxygen source through an external oxygen supply conduit to the baby's mouth and nose areas of the baby's head. The baby pacifier preferably includes a peripheral oxygen deflector that extends generally radially outwardly for deflecting supplemental oxygen emitted from the oxygen delivery holes toward the baby's mouth and nose areas. The baby pacifier preferably includes a medication administering sponge to which a medication solution may be added such that when supplemental oxygen passes therethrough, the medication solution is gradually evaporated delivering medication vapors to the baby's nose area.

The baby pacifier includes a housing that housing includes a front surface adapted to be symmetrically disposed about the baby's head at the mouth and nose areas, and a resilient nipple. The nipple has a bulbous portion and a dependent stem, the stem being attached centrally to the front surface with the nipple extending therefrom. The housing has an interior chamber into which supplemental oxygen is directed through an oxygen inlet opening of a barbed elbow or stem into the interior chamber. The oxygen inlet opening is coupled to the external oxygen source using an external oxygen supply conduit. A plurality of oxygen delivery holes are generally symmetrically spaced laterally about the baby's mouth and nose area. The oxygen delivery holes extend from the interior chamber through the housing about the baby's mouth and nose areas to allow supplemental oxygen to flow outwardly from the interior chamber. The nipple pacifies the baby during suckling while centering the baby pacifier about the baby's head at the mouth and nose areas. The oxygen delivery holes regulate the flow of supplemental oxygen which is gently delivered to the baby's nose area for breathing without requiring invasive oxygen delivery tubes whenever the nipple is in the baby's mouth. The interior chamber and oxygen delivery holes assure an equal distribution of supplemental oxygen from the external oxygen source is conveyed to the nose and mouth areas of the baby.

2. Description of the Prior Art

Babies and other infants (hereinafter all referred to as babies) are increasingly surviving premature births with immature organ formation due to advances in modern medical technology. One of the organs which is particularly fragile in such premature babies are the lungs, which fully mature only late in a pregnancy. Such premature babies may be placed on positive pressure ventilators that assist the lungs in breathing, but ventilators can also damage to the tiny alveoli of the baby's lungs. This may require further supplemental oxygen therapy once the premature baby's lungs develop enough that the ventilator can be discontinued, such as placement of the baby in oxygenated incubators or that oxygen hoods be placed around their heads. This limits the accessibility to the baby and does not provide supplemental oxygen when the baby must be removed from the incubator or hood for feeding, changing, and medical examination.

Oxygen cannulae placed in the baby's nostrils and face masks have been used to provide supplemental oxygen during such periods of removal. However, providing supplemental oxygen to an baby is difficult because the baby often struggles when the cannulae or face mask are secured around the baby's face. Cannulae may also transmit undesirable pressure to the baby's lungs causing lung damage. Furthermore, while it is desirable to place a nipple in the baby's mouth when changing the baby and during medical examination to calm the baby, this may cause the baby to have greater difficulty exhaling due to the increased lung pressure caused by the oxygen cannulae. A smooth, constant flow of supplemental oxygen directed in front of the baby's nose area is the ideal and safest way to administer supplemental oxygen while the baby is nursing.

There have been developed various oxygenating devices for administering supplemental oxygen, some of which include a nipple. Others are adapted to connect to external nipples such as on a nursing bottle. An oxygenating pacifier is disclosed in Press, U.S. Pat. No. 5,375,593 issued on Dec. 27, 1994. Press discloses a pacifier for connection to an external oxygen source, having a nipple, a mouth plate, a housing assembly, and a pair of nasal cannulae coupled to one another extending upwardly out of the housing. The nasal cannulae are positioned such that the nasal cannulae are automatically directed to the baby's nostrils whenever the baby has the nipple in mouth. In one embodiment a bladder is interposed between the nasal cannulae and the external oxygen source for more even distribution of supplemental oxygen from the external oxygen source. In another embodiment the nasal cannulae split in a “T” or a “Y” connection directly coupled to the external oxygen source. The pacifier has the same problems discussed above for pacifiers used with cannulae.

Battaglia et al., U.S. Pat. No. 4,569,461 issued on Jun. 2, 1987 reveals a device for administrating supplemental oxygen to babies during nursing. The device includes an oxygen flow-directing member of a half-funnel shape which includes an inlet opening for connection to an oxygen supply tube, an internal plate having a plurality of holes for supplemental oxygen flow regulation, and a semicircular outlet. The flow-directing member is attachable in one configuration to the neck of a standard nursing bottle using a foam collar with adhesive tabs. The flow-directing member may pivot forward and backward about a pair of rivets connected to the foam collar for directing the supplemental oxygen flow upon the nursing baby's nostrils. In another configuration, the flow-directing member is attachable to a woman's breast using an adhesive pad. A single rivet secures the flow-directing member to the adhesive pad for adjustable direction of the supplemental oxygen flow upon the nursing baby's nostrils. In each configuration, supplemental oxygen is dispensed in the vicinity of the baby's nose and the flow-directing member is pivotable to vary the direction of the supplemental oxygen flow. However, the device requires attachment to a bottle or to the nursing mother's breast to operate. It is not designed to provide supplemental oxygen to the baby other than during nursing.

It is another object of the present invention to provide an baby oxygenation device that provides equal low pressure distribution of supplemental oxygen to each nostril of the child.

It is thus an object of the present invention to provide an baby oxygenation device which may administer medications with the supplemental oxygen.

It is a still further object of the present invention to provide an baby oxygenation device which is readily accepted by the baby without a struggle.

### SUMMARY OF THE INVENTION

The present invention accomplishes the above-stated objectives, as well as others, as may be determined by a fair reading and interpretation of the entire specification.

A baby pacifier is provided for insertion into the mouth of a baby for gently feeding supplemental oxygen supplied from an external oxygen source through an external oxygen supply conduit to the baby's mouth and nose areas of the baby's head including a housing and a nipple. The housing includes a front surface adapted to be symmetrically disposed about the baby's head at the mouth and nose areas. The housing has an interior chamber into which supplemental oxygen is directed through an oxygen inlet opening disposed through the housing into the interior chamber and arranged to be coupled to the external oxygen source using the external oxygen supply conduit. A plurality of oxygen delivery holes are generally symmetrically spaced laterally about the baby's mouth and nose area and which extend from the interior chamber through the housing about the baby's mouth and nose areas to allow supplemental oxygen to flow outwardly from the interior chamber. The nipple has a bulbous portion and a dependent stem, the stem being attached centrally to the front surface with the nipple extending therefrom. The nipple pacifies the baby during suckling while centering the baby pacifier about the baby's head at the mouth and nose areas such that the oxygen delivery holes regulate the flow of supplemental oxygen which is gently delivered to the baby's nose area for breathing without requiring invasive oxygen delivery tubes whenever the nipple is in the baby's mouth. The interior chamber and oxygen delivery holes assure an equal distribution of supplemental oxygen from the external oxygen source is conveyed to the nose and mouth areas of the baby. The baby pacifier may have a housing which comprises respective front and rear housing halves which connect together to form the interior chamber. The housing may includes a peripheral oxygen deflector that extends generally radially outwardly from the housing adapted for deflecting supplemental oxygen emitted from the oxygen delivery holes toward the baby's mouth and nose areas. The baby pacifier may include a medication administering sponge that is positionable within the interior chamber, to which a medication solution may be added such that when the supplemental oxygen passes therethrough, the medication solution is gradually evaporated prior to passing through the oxygen delivery holes delivering medication vapors to the baby's nose area.

### BRIEF DESCRIPTION OF THE DRAWINGS

Various other objects, advantages, and features of the invention will become apparent to those skilled in the art from the following discussion taken in conjunction with the following drawings, in which:

FIG. 1 is a perspective view of a first preferred embodiment of the baby pacifier with gentle oxygen feed, as attached to an oxygen delivery tube.

FIG. 2 is a perspective view of the baby pacifier but without the oxygen delivery tube.

FIG. 3 is an exploded perspective view substantially as in FIG. 2, showing the front and rear housing halves separated, with the barbed elbow for delivering supplemental oxygen to the interior chamber and the oxygen delivery holes leading from the interior chamber.

FIG. 4 is a perspective view of a second preferred embodiment of the baby pacifier with gentle oxygen feed which includes a peripheral oxygen deflector.

FIG. 5 is a perspective view of the baby pacifier from another angle.

FIG. 6 is an exploded perspective view substantially as in FIGS. 4 and 5, showing the front and rear housing halves separated, with the barbed elbow for delivering supplemental oxygen to the interior chamber and the oxygen delivery holes leading from the interior chamber.

FIG. 7 is a perspective view of a third preferred embodiment of the baby pacifier with gentle oxygen feed which delivers medication with the supplemental oxygen.

FIG. 8 is a perspective view of the baby pacifier, showing the upper and lower housing that disconnects from the upper housing to access the sponge which contains medication solution.

FIG. 9 is an exploded perspective view substantially as in FIGS. 7 and 8, showing the interior chamber that contains the sponge.

FIG. 10 is a bottom perspective view of the baby pacifier, the lower housing being removed, showing the oxygen delivery holes which extend from the interior chamber.

FIG. 11 is a side perspective view of the baby pacifier in partial longitudinal cross-section, showing the interior chamber of the nipple, the sponge within the interior chamber of the housing, the oxygen delivery holes which extend from the interior chamber through the upper housing, the threaded connection of the lower housing to the upper housing, the stopper for closing the interior chamber of the housing, and the stem for attaching an oxygen delivery tube.

FIG. 12 is an exploded perspective view in partial longitudinal cross-section substantially as in FIG. 11, showing medication solution being added to the sponge in the interior chamber of the housing from a squeeze bottle.

### DETAILED DESCRIPTION OF THE PREFERRED EMBODIMENTS

As required, detailed embodiments of the present invention are disclosed herein, however, it is to be understood that the disclosed embodiments are merely exemplary of the invention which may be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure.

Reference is now made to the drawings, wherein like characteristics and features of the present invention shown in the various FIGURES are designated by the same reference numerals.

### First Preferred Embodiment

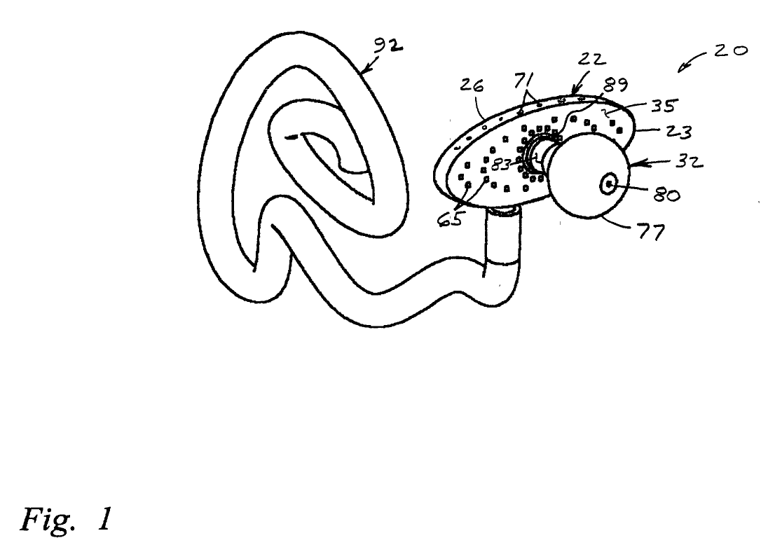

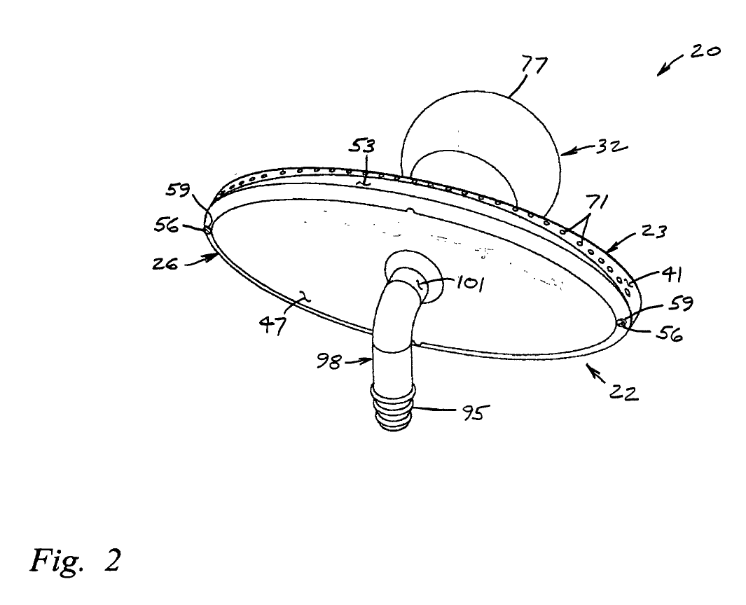

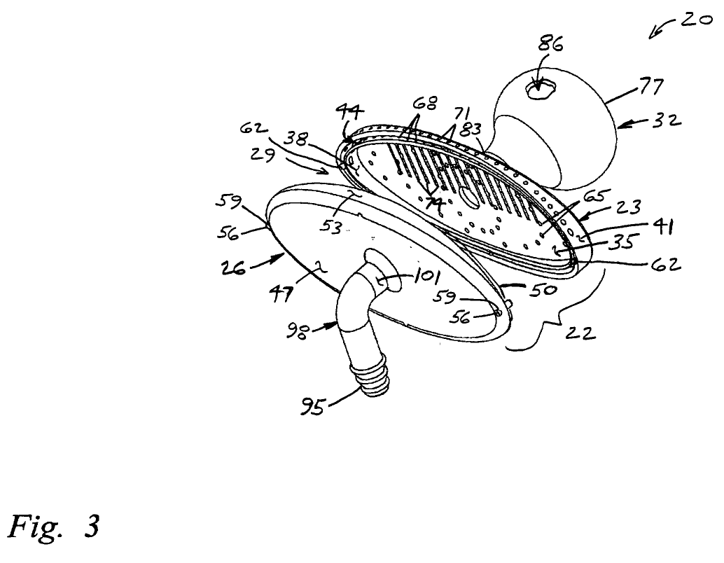

Referring to FIGS. 1-3, a first preferred embodiment of the baby pacifier with gentle oxygen feed 20 is disclosed. The baby pacifier 20 includes a hollow housing 22 comprised of respective front and rear housing halves 23 and 26 which connect together to form an interior chamber 29, and a resilient nipple 32. Front and rear housings 23 and 26 are preferably injection molded from a rigid or semi-rigid plastic such as nylon, polypropylene, polyethylene, or polyvinyl chloride. Nipple 32 is preferably molded from soft latex rubber or a similar non-toxic resilient material. Front housing 23 includes a front wall 35 of a generally oval shape, with respective annular peripheral inner and outer walls 38 and 41 rearwardly dependent therefrom forming an annular space 44 therebetween. Rear housing 26 includes a rear wall 47 of a generally oval shape, with respective annular peripheral inner and outer walls 50 and 53 forwardly dependent therefrom. When assembled together, outer walls 41 and 53 abut with inner wall 50 closely fitting within annular space 44 providing a seal and to key rear housing 26 to front housing 23. Front and rear housings 23 and 26 connect together using a pair of screws 56 that extend through respective clearance holes 59 through rear housing 26 and thread into respective threaded holes 62 in front housing 23. Alternatively, mating pins and holes or similar devices may be used. A plurality of oxygen delivery holes 65, 68, and 71 respectively extend through front wall 35, inner wall 38, and outer wall 41 to allow supplemental oxygen to flow outwardly from interior chamber 29. A plurality of reinforcement ribs 74 extend from front wall 35. Nipple 32 includes a bulbous portion 77 with a central outlet hole 80, and a dependent stem 83 that define an interior chamber 86. Stem 80 snugly fits over a tube 89 that extends outwardly from front housing 23 interconnecting the interior chambers 29 and 86, and outlet hole 80. Supplemental oxygen is introduced into the interior chamber 29 from an oxygen supply device (not shown), through an oxygen delivery tube 92 connected to a barbed end 95 of a tubular elbow 98 of rear housing 26 which connects to interior chamber 29 at a fixed end 101.

The baby pacifier 20 is used by connecting the oxygen delivery tube 92 from the oxygen supply device to the barbed end 95 of the tubular elbow 98, and placing the nipple 32 into the baby's mouth. The supplemental oxygen circulates within the interior chamber 29, and gently flows outwardly through the oxygen delivery holes 65 and 71 at low pressure. The supplemental oxygen flow is evenly distributed throughout all of the oxygen delivery holes 65 and 71 to form a “cloud” of oxygenated air at the baby's nose area which places no additional pressure on the baby's lungs over ambient. The baby is quieted and contented by sucking on the nipple 32 while the supplemental oxygen is gently supplied.

### Second Preferred Embodiment

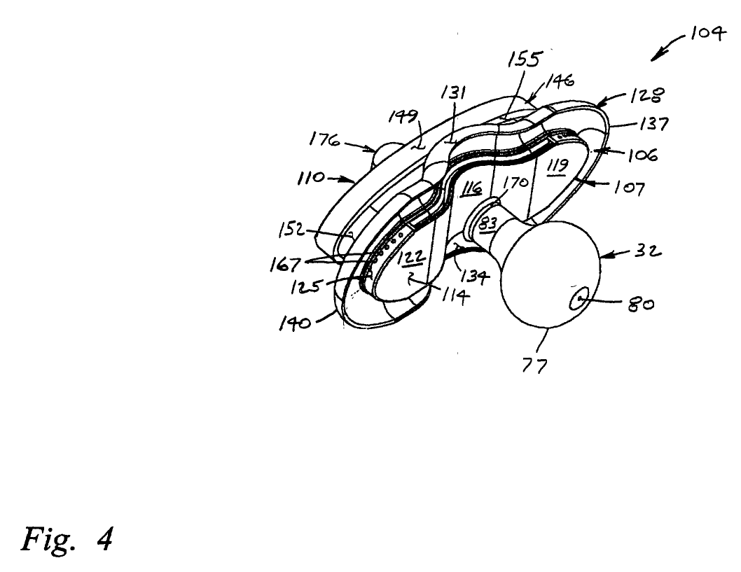

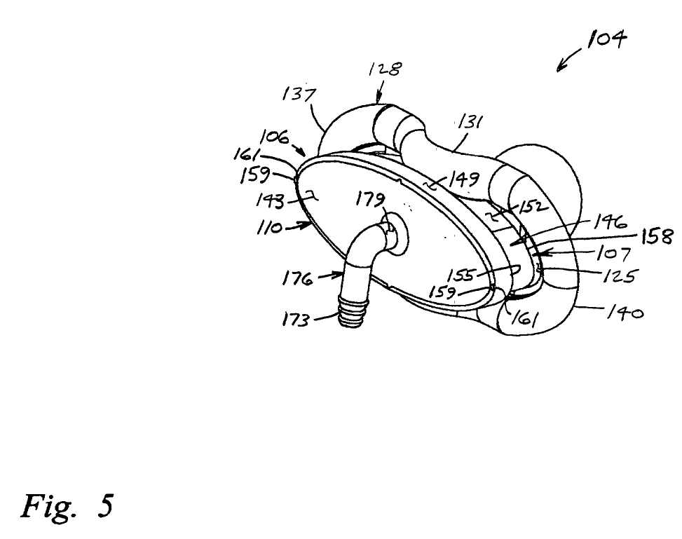

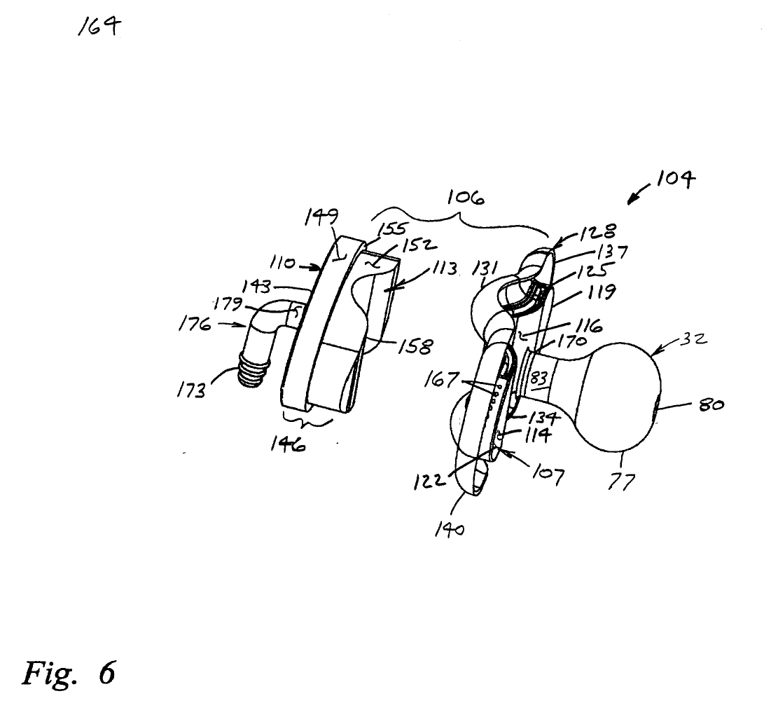

Referring to FIGS. 4-6, a second preferred embodiment of the baby pacifier with gentle oxygen feed 104 is disclosed. The baby pacifier 104 includes a hollow housing 106 comprised of respective front and rear housing halves 107 and 110 which connect together to form an interior chamber 113, and the resilient nipple 32. Front and rear housings 107 and 110 are preferably injection molded from a rigid or semi-rigid plastic such as nylon, polypropylene, polyethylene, or polyvinyl chloride. Front housing 107 includes a front wall 114 of a generally oval shape, having a curved central portion 116 and respective outwardly disposed flat wing portions 119 and 122. An annular peripheral outer wall 125 is rearwardly dependent from front wall 114. Front housing 107 further includes a peripheral oxygen deflector 128 that extends radially from front wall 114, of a half-tube cross-section, that includes respective curved upper and lower central portions 131 and 134, and respective flat wing portions 137 and 140. Rear housing 110 includes a rear wall 143 of a generally oval shape, with an annular peripheral stepped outer wall 146 dependent therefrom comprising respective rearward and forward peripheral walls 149 and 152 interconnected by a connecting wall 155. When assembled together, the forward peripheral wall 152 closely fits about the peripheral outer wall 125 of the front housing 107, which includes a front edge 158 that is curved to match front wall 114, to seal and key rear housing 110 to front housing 107. Front and rear housings 107 and 110 connect together using a pair of screws 159 that extend through respective clearance holes 161 through rear housing 110 and thread into respective threaded holes 164 in front housing 107. Alternatively, mating pins and holes or similar devices may be used. A plurality of oxygen delivery holes 167 extend through the annular peripheral outer wall 125 to allow supplemental oxygen to flow outwardly from interior chamber 113. The stem 83 of nipple 32 snugly fits over a tube 170 that extends outwardly from front housing 107 interconnecting the interior chambers 113 and 86, and outlet hole 80. Supplemental oxygen is introduced into the interior chamber 113 from an oxygen supply device (not shown), through an oxygen delivery tube (not shown) connected to a barbed end 173 of a tubular elbow 176 of rear housing 110 which connects to interior chamber 113 at a fixed end 179.

The baby pacifier 20 is used by connecting the oxygen delivery tube 92 from the oxygen supply device to the barbed end 173 of the tubular elbow 176, and placing the nipple 32 into the baby's mouth. The supplemental oxygen circulates within the interior chamber 113, and gently flows outwardly through the oxygen delivery holes 167 at low pressure. The supplemental oxygen flow is evenly distributed throughout all of the oxygen delivery holes 167 to form a “cloud” of oxygenated air at the baby's nose area, which is further contained by the peripheral oxygen deflector 128. No additional pressure is placed on the baby's lungs over ambient. The baby is quieted and contented by sucking on the nipple 32 while the supplemental oxygen is gently supplied.

### Third Preferred Embodiment

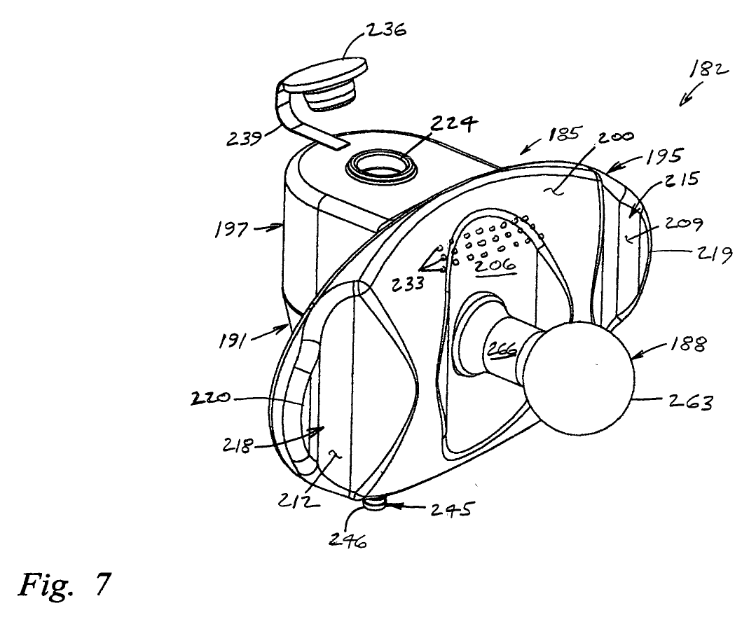

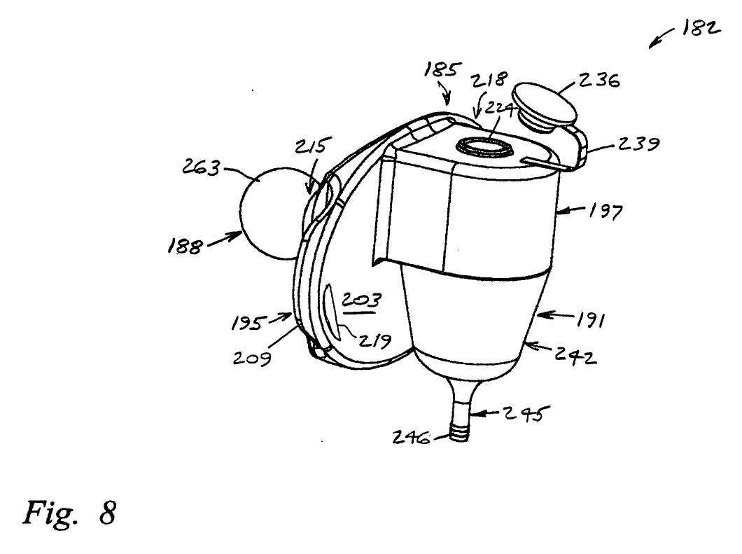

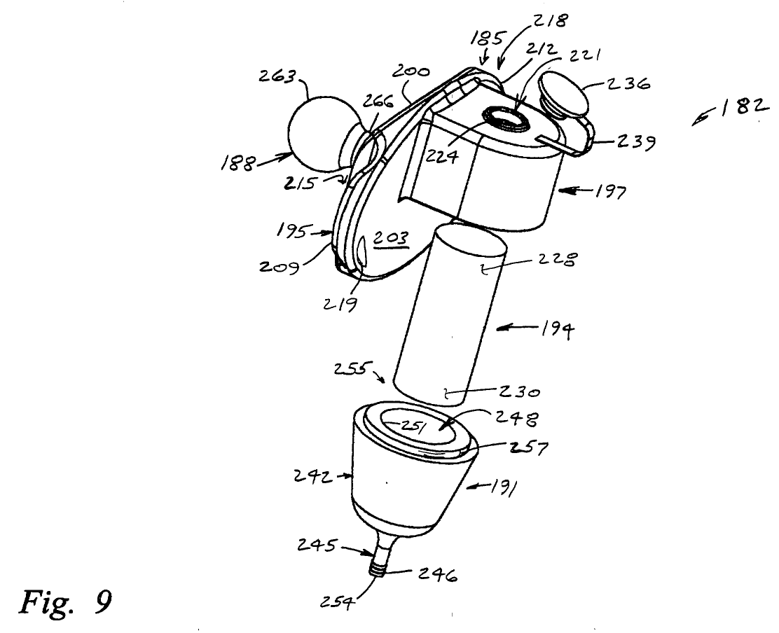

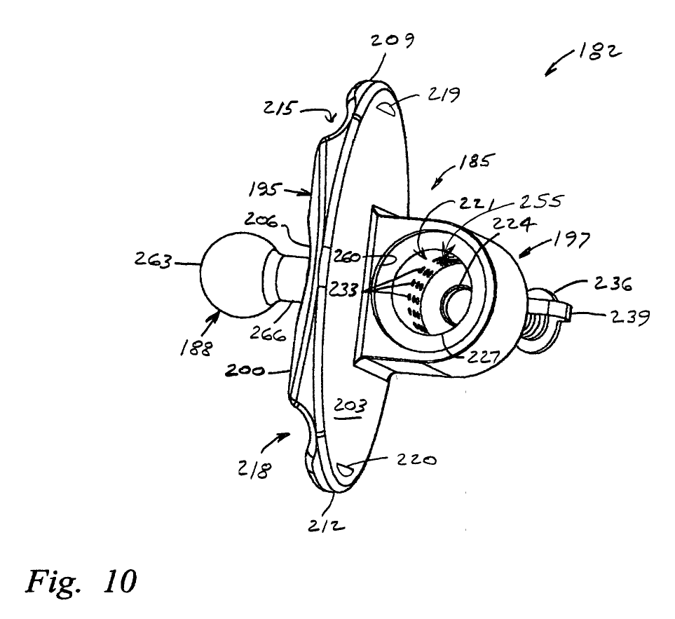

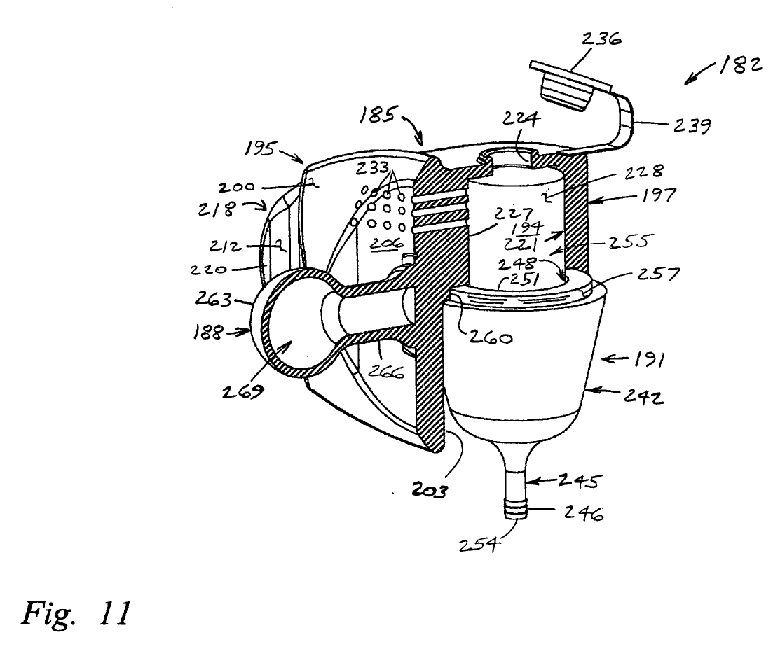

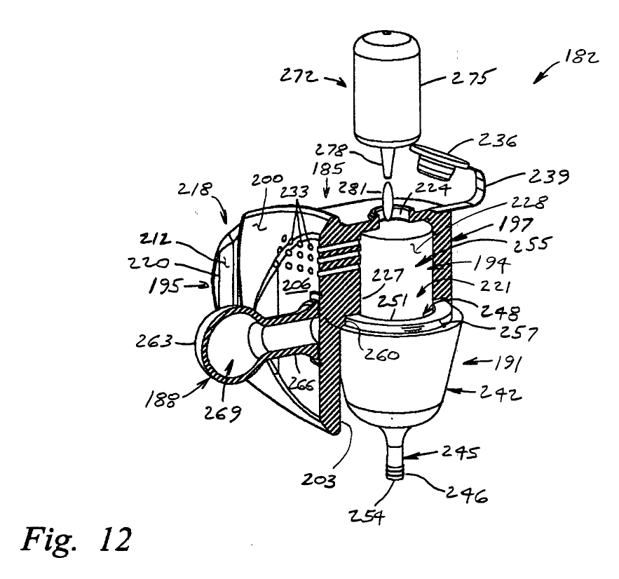

Referring to FIGS. 7-12, a third preferred embodiment of the baby pacifier with gentle oxygen feed 182 is disclosed. The baby pacifier 182 includes an integral upper housing 185 and resilient nipple 188, a lower housing 191, and a cylindrical medication administering sponge 194. Upper housing 185, nipple 188, and lower housing 191 are preferably injection molded from a resilient material such as rubber or a similar non-toxic resilient material. Alternatively, upper and lower housings 185 and 191 may be molded from a harder plastic such as nylon, polypropylene, polyethylene, polyvinyl chloride, or other such material, and the nipple 32 attached to upper housing 185 in a similar manner to baby pacifiers 20 and 104. Spongel94 is preferably made of an open cell polyurethane foam or similar non-toxic material. Absorbent paper materials or the like may also be used. Housing 185 includes a front portion 195 of a generally oval shape, and a rear portion 197 of a rounded rectangular shape. Front portion 195 has a curved front surface 200 and a flat rear surface 203. Front surface 200 includes a concave central portion 206 and a pair of outwardly disposed wing portions 209 and 212 having respective end depressions 215 and 218. A pair of strap connection slots 219 and 220 extend through the respective wing portions 209 and 212 at the end depressions 215 and 218. The strap connection slots 219 and 220 may be used to attach an adjustable strap (not shown) to retain the pacifier 182 to the baby's mouth area should the baby not retain it by sucking. A vertical bore 221 extends through rear portion 197, having a small inlet bore 224 and a large sponge receiving bore 227 into which an upper portion 228 of sponge 194 closely fits, with a lower portion 230 of sponge 194 extending downwardly therefrom. A plurality of oxygen delivery holes 233 extend longitudinally forwardly from bore 221 through front portion 195 of upper housing 185 to allow supplemental oxygen to flow outwardly from bore 221. A stopper 236 is integrally molded to rear portion 197 through a connecting strap 239 to allow selective closing of the inlet bore 224.

Lower housing 191 is of an annular, slightly downwardly tapered shape, having a body 242 and a downwardly dependent stem 245 having a barbed free end 246. A vertical bore 248 extends through body 242, having a large sponge receiving bore 251 into which the lower portion 230 of sponge 194 closely fits, and a small inlet bore 254 which extends through stem 245. Bores 221 and 248 comprise an interior chamber 255. Lower housing 191 removably connects to upper housing 185 through mating externally and internally threaded portions 257 and 260 thereof. A resilient seal (not shown) may be included between the upper and lower housings 185 and 191 for positive gas and liquid sealing therebetween. Alternatively, the upper and lower housings 185 and 191 may be permanently affixed together to contain sponge 194 such as by using heat fusion or adhesives. Nipple 188 is similar to nipple 32, having a bulbous portion 263, and a dependent stem 266 that define an interior chamber 269. Stem 266 extends from the central portion 206 of upper housing 185, being integrally molded therewith. Supplemental oxygen is introduced into the interior chamber 255 from an oxygen supply device (not shown), through an oxygen delivery tube (not shown) connected to the barbed free end 246 of stem 245.

The baby pacifier 182 is used by removing the stopper 236 from the inlet bore 224, and soaking the sponge 194 using a squeeze bottle 272. The body 275 of bottle 272 is squeezed with the stem 278 disposed above the inlet bore 224 such that a medication solution 281 flows onto sponge 194. The oxygen delivery tube 92 from the oxygen supply device is then connected to the barbed end 246 of the tubular stem 245, and the nipple 188 placed into the baby's mouth. The supplemental oxygen circulates within the interior chamber 255 through and about the sponge 194 to evaporate some of the medication solution 281, and gently flows outwardly through the oxygen delivery holes 233 at low pressure. The supplemental oxygen flow is evenly distributed throughout all of the oxygen delivery holes 233 to form a “cloud” of medicated oxygenated air at the baby's nose area. No additional pressure is placed on the baby's lungs over ambient. The baby is quieted and contented by sucking on the nipple 188 while the supplemental oxygen is gently supplied.

While the invention has been described, disclosed, illustrated and shown in various terms or certain embodiments or modifications which it has assumed in practice, the scope of the invention is not intended to be, nor should it be deemed to be, limited thereby and such other modifications or embodiments as may be suggested by the teaching herein are particularly reversed especially as they fall within the breadth and scope of the claims here appended.

I claim as my invention:

1. A baby pacifier for insertion into the mouth of a baby for gently feeding supplemental oxygen supplied from an external oxygen source through an external oxygen supply conduit to the baby's mouth and nose areas of the baby's head, comprising:
a housing that includes a front surface adapted to be symmetrically disposed about the baby's head at the mouth and nose areas, said housing having an interior chamber into which supplemental oxygen is directed through an oxygen inlet opening disposed through said housing into said interior chamber and arranged to be coupled to the external oxygen source using the external oxygen supply conduit, and a plurality of oxygen delivery holes generally symmetrically spaced laterally about the baby's mouth and nose area and which extend from said interior chamber through said housing about the baby's mouth and nose areas to allow supplemental oxygen to flow outwardly from said interior chamber;
a nipple having a bulbous portion and a dependent stem, said stem being attached centrally to said front surface with said nipple extending therefrom; and
wherein said nipple pacifies the baby during suckling while centering the baby pacifier about the baby's head at the mouth and nose areas such that said oxygen delivery holes regulate the flow of supplemental oxygen which is gently delivered to the baby's nose area for breathing without requiring invasive oxygen delivery tubes whenever said nipple is in the baby's mouth, said interior chamber and oxygen delivery holes which assure an equal distribution of supplemental oxygen from the external oxygen source is conveyed to the nose and mouth areas of the baby.

2. The baby pacifier of claim 1, wherein the housing comprises respective front and rear housing halves which connect together to form the interior chamber.

3. The baby pacifier of claim 2, wherein the front and rear housing halves include respective front and rear walls of a generally rounded shape, with at least one thereof having a peripheral outer wall dependent therefrom adapted to closely engage opposing of said housing halves.

4. The baby pacifier of claim 3, wherein at least some of the oxygen delivery holes extend upwardly through the peripheral outer wall to expel supplemental oxygen adjacent the nose area of the baby's head.

5. The baby pacifier of claim 3, wherein the front and rear housing halves are matingly keyed together sealing said front and rear housing halves together.

6. The baby pacifier of claim 2, wherein the front and rear housing halves connect together using a plurality of screws.

7. The baby pacifier of claim 2, wherein the bulbous portion and dependent stem of the nipple define an interior chamber, and said bulbous portion having a central outlet hole.

8. The baby pacifier of claim 7, wherein the interior chamber of the nipple is connected to the interior chamber of the housing by a nipple hole through the front housing half such that supplemental oxygen may flow through both interior chambers and out through the central outlet hole of said nipple.

9. The baby pacifier of claim 7, wherein the stem of the nipple snugly fits over an outlet tube that extends outwardly from the front housing half.

10. The baby pacifier of claim 2, wherein supplemental oxygen is introduced into the interior chamber through a free end of an oxygen inlet tube for connection to the oxygen supply conduit with a fixed end thereof connected to the housing about the oxygen inlet hole.

11. The baby pacifier of claim 10, wherein the oxygen inlet tube comprises an elbow.

12. The baby pacifier of claim 10, wherein the free end of the oxygen inlet tube is barbed.

13. The baby pacifier of claim 2, wherein the oxygen delivery holes extend through the front wall.

14. The baby pacifier of claim 2, wherein the front housing half further includes a peripheral oxygen deflector that extends generally radially outwardly from the front housing half adapted for deflecting supplemental oxygen emitted from the oxygen delivery holes toward the baby's mouth and nose areas.

15. The baby pacifier of claim 14, wherein the front housing half includes a front wall having a concave central portion and respective outwardly disposed wing portions, and wherein the peripheral oxygen deflector includes respective concave upper and lower central portions, and respective flat wing portions corresponding to said front housing half.

16. The baby pacifier of claim 15, wherein the front housing half includes a peripheral outer wall curved to follows the front wall, and wherein the rear housing half includes a rear wall and a peripheral stepped outer wall dependent therefrom comprising respective rearward and forward peripheral walls interconnected by a connecting wall, said forward peripheral wall having a front edge that is curved to closely match front wall.

17. The baby pacifier of claim 14, wherein the front and rear housing halves include respective front and rear walls of a generally rounded shape, with at least one thereof having a peripheral outer wall dependent therefrom adapted to closely engage opposing of said housing halves, and wherein at least some of the oxygen delivery holes extend upwardly through said peripheral outer wall to expel supplemental oxygen adjacent the nose area of the baby's head.

18. The baby pacifier of claim 14, wherein the peripheral oxygen deflector is of generally a half-tube cross-section at the front housing half.

19. The baby pacifier of claim 1, additionally comprising a medication administering sponge that is positionable within the interior chamber to which a medication solution may be added such that when the supplemental oxygen passes therethrough, the medication solution is gradually evaporated prior to passing through the oxygen delivery holes delivering medication vapors to the baby's nose area.

20. The baby pacifier of claim 19, wherein the housing comprises two separate housings which removably connect together to enclose the interior chamber.

21. The baby pacifier of claim 20, wherein one housing comprises an upper housing having a front portion and a rear portion, said rear portion having a vertical bore that extends upwardly partially through the rear portion comprising a large sponge receiving bore into which an upper portion of the sponge closely fits, and wherein one housing comprises a lower housing having a body of a generally cylindrical shape and a downwardly dependent stem with a vertical bore extending through said body and said stem, said vertical bore of said body and stem which includes a large sponge receiving bore into which a lower portion of sponge closely fits, said vertical bore within said stem comprising the oxygen inlet bore.

22. The baby pacifier of claim 21, wherein the lower housing removably threadably connects to upper housing.

23. The baby pacifier of claim 21, wherein the oxygen delivery holes extend longitudinally forwardly from the vertical bore of the rear portion through the front portion of the upper housing to allow supplemental oxygen to flow outwardly from said vertical bore.

24. The baby pacifier of claim 21, wherein a small inlet bore of the vertical bore extends upwardly through the rear portion of the upper housing, and said upper housing includes a stopper that is connected thereto through a connecting strap to allow selective closing of the inlet bore by inserting said stopper thereinto.

25. The baby pacifier of claim 21, wherein the front portion of the upper housing includes a front surface having a concave central portion and respective outwardly disposed wing portions.

26. The baby pacifier of claim 25, wherein the nipple extends from the concave central portion of the front surface.

27. The baby pacifier of claim 21, wherein the upper housing and the nipple are integrally molded of a resilient material.

28. The baby pacifier of claim 1, wherein the housing includes respective strap connection slots disposed at opposite sides thereof for attachment of a retaining strap that is positionable about the baby's head to retain the baby pacifier to the baby's mouth area.

29. A baby pacifier for insertion into the mouth of a baby for gently feeding supplemental oxygen supplied from an external oxygen source through an external oxygen supply conduit to the baby's mouth and nose areas of the baby's head, comprising:
a housing that includes a front surface adapted to be symmetrically disposed about the baby's head at the mouth and nose areas, said housing having an interior chamber into which supplemental oxygen is directed through an oxygen inlet opening disposed through said housing into said interior chamber and arranged to be coupled to the external oxygen source using the external oxygen supply conduit, and a plurality of oxygen delivery holes generally symmetrically spaced laterally about the baby's mouth and nose area and which extend from said interior chamber through said housing about the baby's mouth and nose areas to allow supplemental oxygen to flow outwardly from said interior chamber;
a nipple having a bulbous portion and a dependent stem, said stem being attached centrally to said front surface with said nipple extending therefrom;
a peripheral oxygen deflector that extends generally radially outwardly from said housing adapted for deflecting supplemental oxygen emitted from the oxygen delivery holes toward the baby's mouth and nose areas; and
wherein said nipple pacifies the baby during suckling while centering the baby pacifier about the baby's head at the mouth and nose areas such that said oxygen delivery holes regulate the flow of supplemental oxygen which is gently delivered to the baby's nose area for breathing without requiring invasive oxygen delivery tubes whenever said nipple is in the baby's mouth, said interior chamber and oxygen delivery holes which assure an equal distribution of supplemental oxygen from the external oxygen source is conveyed to the nose and mouth areas of the baby.

30. The baby pacifier of claim 29, wherein the housing includes a front surface having a concave central portion and respective outwardly disposed wing portions, and wherein the peripheral oxygen deflector includes respective concave upper and lower central portions, and respective flat wing portions corresponding to said housing.

31. The baby pacifier of claim 30, wherein at least some of the oxygen delivery holes extend upwardly through a peripheral outer surface of the housing adjacent the front surface to expel supplemental oxygen adjacent the nose area of the baby's head.

32. The baby pacifier of claim 30, wherein the oxygen delivery holes extend through the front wall.

33. A baby pacifier for insertion into the mouth of a baby for gently feeding supplemental oxygen supplied from an external oxygen source through an external oxygen supply conduit to the baby's mouth and nose areas of the baby's head, comprising:
a housing that includes a front surface adapted to be symmetrically disposed about the baby's head at the mouth and nose areas, said housing having an interior chamber into which supplemental oxygen is directed through an oxygen inlet opening disposed through said housing into said interior chamber and arranged to be coupled to the external oxygen source using the external oxygen supply conduit, and a plurality of oxygen delivery holes generally symmetrically spaced laterally about the baby's mouth and nose area and which extend from said interior chamber through said housing about the baby's mouth and nose areas to allow supplemental oxygen to flow outwardly from said interior chamber;
a nipple having a bulbous portion and a dependent stem, said stem being attached centrally to said front surface with said nipple extending therefrom;
a medication administering sponge that is positionable within the interior chamber such that the supplemental oxygen passes therethrough to pick up evaporating medication prior to passing through the oxygen delivery holes delivering medication vapors to the baby's nose area; and
wherein said nipple pacifies the baby during suckling while centering the baby pacifier about the baby's head at the mouth and nose areas such that said oxygen delivery holes regulate the flow of supplemental oxygen which is gently delivered to the baby's nose area for breathing without requiring invasive oxygen delivery tubes whenever said nipple is in the baby's mouth, said interior chamber and oxygen delivery holes which assure an equal distribution of supplemental oxygen from the external oxygen source is conveyed to the nose and mouth areas of the baby.

34. The baby pacifier of claim 33, wherein a small inlet bore for adding a medicating solution extends from the interior chamber outwardly through the housing, and said housing includes a stopper that is connected thereto through a connecting strap to allow selective closing of the inlet bore following addition of the medicating solution by inserting said stopper thereinto.

35. The baby pacifier of claim 33, wherein the front surface includes a concave central portion and respective outwardly disposed wing portions, with the nipple extending from said concave central portion of said front surface.

36. The baby pacifier of claim 33, wherein the housing includes respective strap connection slots disposed at opposite sides thereof for attachment of a retaining strap that is positionable about the baby's head to retain the baby pacifier to the baby's mouth area.
